(19) United States
(12) Patent Application Publication
(10) Pub. No.: US 2024/0197515 A1
(43) Pub. Date: Jun. 20, 2024

(54) INVOLUNTARY MOVEMENT DAMPENING DEVICE

(71) Applicant: Chlidren's Hospital of Orange County, Orange, CA (US)

(72) Inventors: Sharief Khalil TARAMAN, Laguna Nigel, CA (US); Jason Ivey, Anaheim, CA (US); Tianyi Li, Laguna Hills, CA (US); Amy Lynn Moss, San Marcos, CA (US); Quang Trong Nguyen, Irvine, CA (US); Kimmai Tana Phan, Atlanta, GA (US); Adriana Rodriguez, Irvine, CA (US); Salma Waleed Serhal, Fullerton, CA (US); Taylor Loren White, Oak View, CA (US); Dylan Thomas Law, San Clemente, CA (US); Benjamin Reyes Dadacay, Long Beach, CA (US); Myong Kim, Tustin, CA (US)

(21) Appl. No.: 18/555,721

(22) PCT Filed: Apr. 15, 2022
(86) PCT No.: PCT/US22/25121

§ 371 Date: Oct. 16, 2023

Related U.S. Application Data
(60) Provisional application No. 63/175,799, filed on Apr. 16, 2021.

(51) Int. Cl. A61F 5/01; A61H 7/00

(52) U.S. Cl. CPC A61F 5/013; A61H 7/001; A61F 2005/0169; A61H 2201/165

(57) ABSTRACT

A involuntary movement dampening device is described that can reduce extremity pain and/or dampen involuntary movement of an extremity. For example, the device can include a first coupling extension including a first extension and a first coupling feature that can secure to a first part of an extremity of a user. The device can include a second coupling extension including a second extension and a second coupling feature that can secure to a second part of the extremity of the user. The device can include an adjustable hinge that provides a pivotable coupling between the first extension and the second extension. The adjustable hinge can provide more than one dampening settings each providing a different level of pivoting resistance between the first extension and the second extension to thereby at least dampen involuntary pivoting between the first part and the second part of the extremity.

### CROSS-REFERENCE TO RELATED APPLICATION

The current application claims priority under 35 U.S.C. § 119(e) to U.S. Provisional patent application Ser. No. 63/175,799, filed on Apr. 16, 2021 and entitled “Involuntary Movement Dampening Device,” which is incorporated by reference herein in its entirety.

### TECHNICAL FIELD

The subject matter described herein relates to various embodiments of an involuntary movement dampening device and related methods.

### BACKGROUND

Ataxic cerebral palsy (ACP) is a neuromuscular disorder caused by damage to the motor cortex during fetal development. ACP is a rare form of cerebral palsy affecting approximately 10% of all diagnosed cases. There is no cure for ACP and current treatments have shown little promise.

A person with ACP can suffer from tremors along one or more extremities (e.g., arms, legs), which can interfere with performing Activities of Daily Living (ADLs), such as eating, dressing, writing and typing. At least some wearable orthotics have been used to try and assist people with ACP but they either do not provide sufficient support or are not user-friendly (e.g., are too bulky, lock the joint along the extremity, etc.). Other physical disabilities can cause people to experience involuntary movements, including tremors, along one or more extremities, which can significantly impair ADLs. At least reducing or dampening such involuntary movements can improve a person's wellbeing and ability to perform ADLs.

### SUMMARY

Aspects of the current subject matter can include various embodiments of an involuntary movement dampening device that can reduce extremity pain and/or dampen involuntary movement of an extremity. In one aspect, an involuntary movement dampening device can include a first coupling extension including a first extension and a first coupling feature positioned along the first extension. The first coupling feature can be configured to secure to a first part of an extremity of a user. The involuntary movement dampening device can include a second coupling extension including a second extension and a second coupling feature positioned along the second extension. The second coupling feature can be configured to secure to a second part of the extremity of the user. The involuntary movement dampening device can include an adjustable hinge that provides a pivotable coupling between the first extension and the second extension. The adjustable hinge can provide more than one dampening settings, and each dampening setting of the more than one dampening settings can provide a different level of pivoting resistance between the first extension and the second extension to thereby at least dampen involuntary pivoting between the first part and the second part of the extremity.

In some variations one or more of the following features can optionally be included in any feasible combination. The involuntary movement dampening device can further include a nerve pressure feature positioned along the first coupling feature and/or the second coupling feature for applying a pressure against the ulnar nerve when the associated first coupling feature and/or the second coupling feature is coupled to the extremity. The involuntary pivoting movement is caused by tremors or other involuntary movements experienced along the extremity of the user. The nerve pressure feature can include an extruded material extending from an inner wall of the second coupling feature. The extruded material can include a spherical shape. The nerve pressure feature can be moveable along an inner wall of the second coupling feature. The involuntary movement dampening device can include at least one length adjusting feature for moving the first coupling feature or the second coupling feature closer to or further away from the adjustable hinge. The adjustable hinge can include a friction feature that increases pivoting resistance between the first extension and the second extension as compression of the friction feature is increased within the adjustable hinge. The friction feature can decrease resistance in movement of the first and second extensions as compression of the friction feature decreases within the adjustable hinge.

In some embodiments, the adjustable hinge can be configured to provide at least three different levels of pivoting resistance between the first extension and the second extension. The adjustable hinge can include an adjustment feature that can be rotated by a user to at least three different positions, and each position can configure the adjustable hinge to provide one of the at least three different levels of pivoting resistance. The adjustable hinge can include a sensory feedback mechanism that provides at least one of a tactile feedback and an audible feedback to a user as a result of the adjustable hinge changing to a different level of pivoting resistance. The first and second coupling features can each include an adjustable strap. The first and second coupling features can include an inflatable strap. The second coupling extension can include more than one second coupling feature that are moveable along the second extension relative to each other and relative to the adjustable hinge.

In another interrelated aspect of the current subject matter, a method of an involuntary movement dampening device includes securing an embodiment of the involuntary movement dampening device to an extremity of a user and adjusting a pivoting resistance of an adjustable hinge of the involuntary movement dampening device. The involuntary movement dampening device can include a first coupling extension including a first extension and a first coupling feature positioned along the first extension. The first coupling feature can be configured to secure to a first part of an extremity of a user. The involuntary movement dampening device can include a second coupling extension including a second extension and a second coupling feature positioned along the second extension, and the second coupling feature can be configured to secure to a second part of the extremity of the user. The adjustable hinge can provide a pivotable coupling between the first extension and the second extension. The adjustable hinge can provide more than one dampening setting, and each dampening setting of the more than one dampening settings can provide a different level of pivoting resistance between the first extension and the second extension to thereby at least dampen involuntary pivoting between the first part and the second part of the extremity.

In some embodiments, the method can further include adjusting the pivoting resistance of the adjustable hinge of the involuntary movement dampening device to achieve a second level of pivoting resistance, wherein the second level of pivoting resistance is greater than the first level of pivoting resistance. The dampening of the involuntary movement can be greater as a result the adjustment hinge having the second level of pivoting resistance compared to the adjustment hinge having the first level of pivoting resistance. The adjustment hinge can increase a compression force and friction force to at least one of a first hinge-coupling end of the first extension and/or a second hinge-coupling end of the second extension to thereby increase a pivoting resistance between the first extension and the second extension.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A illustrates an embodiment of an involuntary movement dampening device consistent with implementations of the current subject matter;

FIG. 1B shows an exploded view of the involuntary movement dampening device of FIG. 1A;

FIG. 1C shows an example of the involuntary movement dampening device of FIG. 1A coupled to an arm of a user;

FIG. 1D shows a partial perspective view of a second coupling feature of the involuntary movement dampening device of FIG. 1A showing nerve pressure features along an inner wall of the second coupling feature;

FIG. 2 illustrates another embodiment of the involuntary movement dampening device including adjustable first and second extensions;

FIG. 3A illustrates yet another embodiment of the involuntary movement dampening device including an adjustment feature that provides perceptible feedback;

FIG. 3B shows an exploded view of the involuntary movement dampening device of FIG. 3A; and

FIG. 4 shows a partial perspective view of an embodiment of the second coupling feature including nerve pressure features that are movable along the second coupling feature.

When practical, similar reference numbers denote similar structures, features, or elements.

### DETAILED DESCRIPTION

The current subject matter is directed to an involuntary movement dampening device that can at least provide support, reduce pain and reduce involuntary movement of an extremity of a user. For example, the involuntary movement dampening device can be secured to an extremity of a user, such as an arm or leg, for at least dampening involuntary movements (e.g., tremors) associated with the extremity. Various embodiments of the involuntary movement dampening device are described herein that include an adjustable hinge that is configurable or adjustable to provide more than one level of pivoting resistance to prevent and/or dampen involuntary movements of the associated extremity. Furthermore, at least some embodiments of the involuntary movement dampening device can include a nerve pressure feature that is configured to apply a pressure against the ulnar nerve when the involuntary movement dampening device is being worn on an arm of the user. For example, the pressure applied by the nerve pressure feature against the ulnar nerve can assist with reducing pain and preventing or reducing involuntary movements of the associated extremity, such as tremors (e.g., by encouraging muscle flexion). Various embodiments of the involuntary movement dampening device are described in greater detail below.

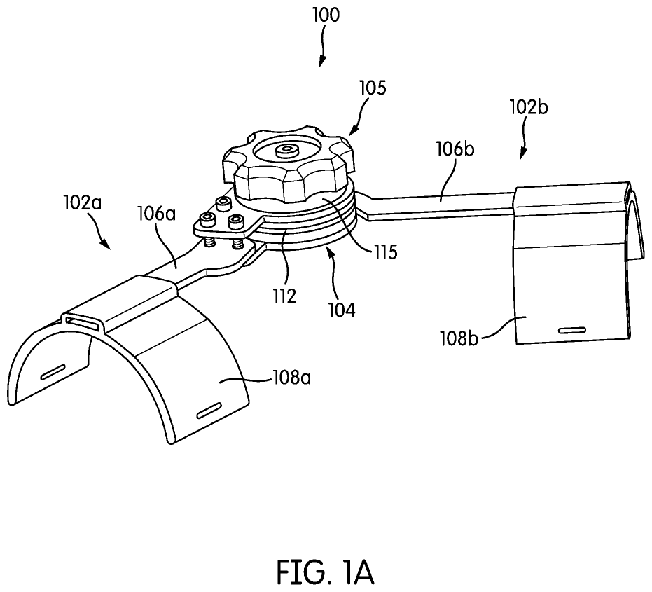

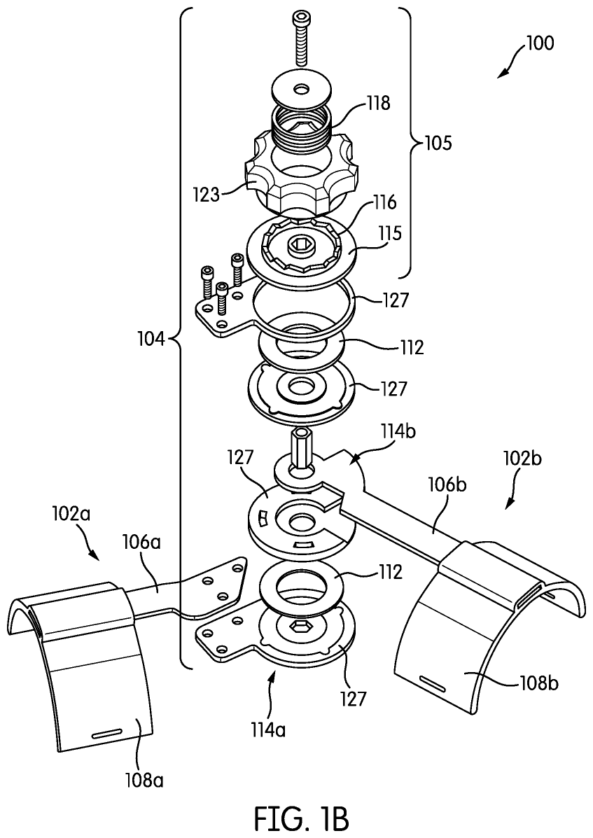

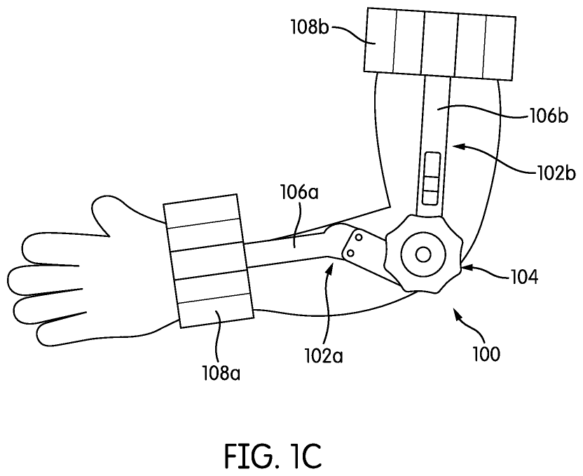

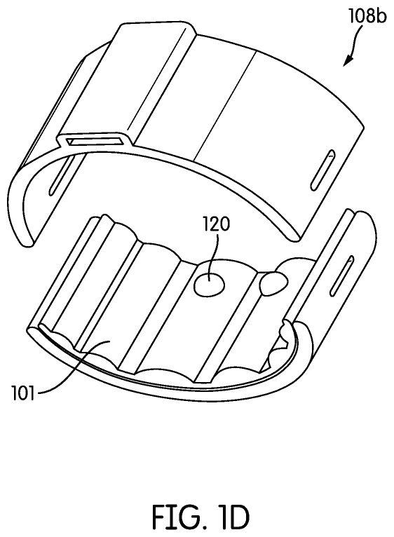

FIGS. 1A-1D illustrate an embodiment of the involuntary movement dampening device 100 including a first coupling extension 102a pivotably coupled to a second coupling extension 102b via an adjustable hinge 104. The first coupling extension 102a can include a first extension 106a and a first coupling feature 108a positioned along the first extension 106a. The second coupling extension 102b can include a second extension 106b and a second coupling feature 108b positioned along the second extension 106b. For example, the first coupling feature 108a can be configured to couple to a first part (e.g., a distal portion) of an extremity of a user. Additionally, the second coupling feature 108b can be configured to couple to a second part (e.g., a proximal portion) of the extremity of the user. For example, the first part of an extremity can be a position along the extremity that is on a first side of a joint (e.g., knee, elbow), and the second part of the same extremity can be along a second side of the same joint. As such, the involuntary movement dampening device 100, if coupled to an arm, can have the first coupling feature 108a coupled to a lower arm, forearm and/or wrist and the second coupling feature 108b coupled to an upper arm and/or around a bicep. Such coupling of the first and second coupling features 108a and 108b can allow the involuntary movement dampening device 100 to limit and/or dampen pivoting of the first part of the extremity relative to the second part of the extremity.

In some embodiments, the first coupling feature 108a and the second coupling feature 108b each include adjustable and releasably securing straps. For example, the first couple feature 108a and the second coupling feature 108b can each include inflatable straps 101, as shown in FIG. 1D, that can be inflated manually or electronically (e.g., battery powered) in order to secure the involuntary movement dampening device 100 onto the extremity. The first coupling feature 108a and the second coupling feature 108b can each include any number of a variety of releasable and adjustable securing features (e.g., Velcro, latches, etc.) for securing the first coupling extension 102a and the second coupling extension 102b to the extremity without departing from the scope of this disclosure.

As shown in FIG. 1C, when the first coupling feature 108a and the second coupling feature 108b are coupled to the first part and second part, respectively, of an arm of the user, the involuntary movement dampening device 100 can assist with controlling movement of the forearm relative to the upper arm, such as for dampening involuntary movements (e.g., tremors) of the arm. For example, such dampening or controlling of movement by the involuntary movement dampening device 100 can include reducing an acceleration of involuntary movement, a frequency of involuntary movement (e.g., distance of involuntary movement over time), and a range or distance of involuntary movement of the extremity. Such reductions in acceleration, frequency, and/or distance of involuntary movement can assist the user in performing more precise movements more easily and with greater accuracy.

In some embodiments of the involuntary movement dampening device 100, the adjustable hinge 104 can be adjustable to provide at least two different levels of resistance or pivoting resistance against movement (e.g., pivoting) between the first coupling extension 102a and the second coupling extension 102a, including between the first extension 106a and the second extension 106b. For example, a level of pivoting resistance can cause complete stabilization and/or prevent movement of the associated joint (e.g., elbow joint), and another level of pivoting resistance can allow at least some movement of the associated joint. Each level of pivoting resistance provided by the adjustable hinge 104 can affect (e.g., increase or decrease) the dampening or controlling of movement of the extremity by the involuntary movement dampening device 100, such as reduce and/or increase the acceleration, frequency, and/or distance of involuntary movement of the extremity, such as a first part of the extremity (e.g., forearm) relative to a second part of the extremity (e.g., upper arm).

For example, the adjustable hinge 104 can include an adjustment feature 105 that, when adjusted, can set different levels of pivoting resistance. In some embodiments, the adjustment feature 105 can include a rotatable knob that, when rotated in a first direction, causes the pivoting resistance to increase and, when rotated in a second direction, causes the pivoting resistance to decrease. For example, a user may want to increase pivoting resistance when performing (or preparing to perform) more precise actions or movements. For example, a user wearing the involuntary movement dampening device 100 along an arm may increase pivoting resistance when using the arm to write, and may decrease the pivoting resistance when not performing precise arm movements and/or not attempting to limit arm movement (e.g., relaxed arms while walking).

For example, different pivoting resistance levels can be achieved by varying an amount of friction and/or compression within the adjustable hinge 104. For example, adjusting the adjustable hinge 104 to increase pivoting resistance can include increasing compression and/or friction within the adjustable hinge 104 to thereby resist pivoting of the first extension 106a and the second extension 106b relative to each other. For example, the adjustment feature 105 can be rotated clockwise to increase pivoting resistance in the adjustable hinge 104 and the adjustment feature 105 can be rotated counter-clockwise to reduce pivoting resistance in the adjustable hinge 104. The involuntary movement dampening device 100 can be efficiently adjusted (e.g., by a user, physician, etc.) to reduce and/or increase pivoting resistance in the adjustable hinge 104 to thereby customize the involuntary movement dampening device 100 to most effectively dampen or decrease various types and strengths of involuntary movements (e.g., tremors) experienced by the user.

A shown in FIG. 1B, the adjustable hinge 104 can include at least one friction feature 112 that can assist with changing the pivoting resistance in the adjustable hinge 104. In some embodiments, the friction feature 112 can include a substantially flat rubber (e.g., neoprene) disc that is compressible. For example, at least one friction feature 112 can be positioned adjacent a first hinge-coupling end 114a of the first extension 106a and/or adjacent a second hinge-coupling end 114b of the second extension 106b. For example, the first hinge-coupling end 114a and/or the second hinge-coupling end 114b can form a part of the adjustable hinge 104 and each include an axis of rotation that is collinear with an axis of rotation of the adjustable hinge 104. As such, a change in compression and/or friction within the adjustable hinge 104 can cause a change in an ability of the first extension 106a and/or second extension 106b to pivot relative to each other.

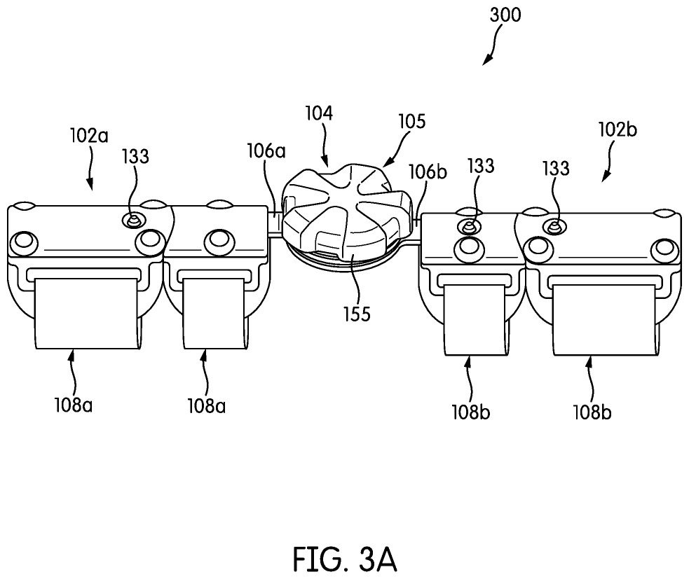

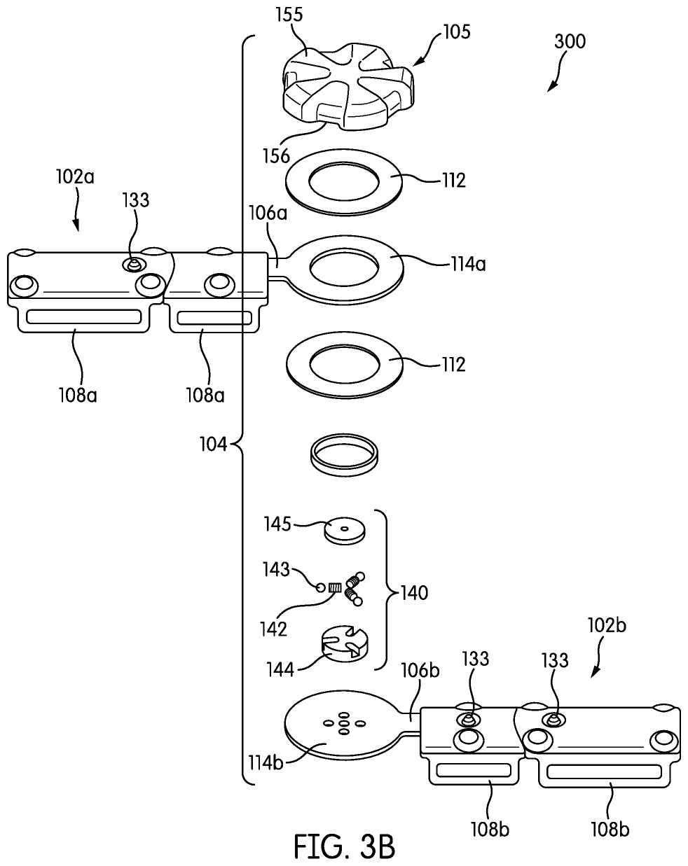

For example, the adjustable hinge 104 can be configured to compress the at least one friction feature 112, such as against the first hinge-coupling end 114a and/or the second hinge-coupling end 114b, to thereby cause the adjustable hinge 104 to increase pivoting resistance between the first and second extensions 106a and 106b, respectively, and thus dampen involuntary movement of the extremity to which the involuntary movement dampening device 100 is coupled to. Additionally, the adjustable hinge 104 can be configured to decompress the at least one friction feature 112 to thereby cause the adjustable hinge 104 to decrease pivoting resistance between the first and second extensions 106a and 106b, respectively, and thus allow more involuntary movement of the extremity to which the involuntary movement dampening device 100 is coupled to. In some embodiments, rotation of the adjustment feature 105 of the adjustable hinge 104 can change the pivoting resistance, such as by causing an increase or decrease in compression and/or resistance within the adjustable hinge 104. As will be described with respect to the involuntary movement dampening device 300 embodiment shown in FIGS. 3A and 3B, some embodiments of the involuntary movement dampening device 100 can include a sensory feedback mechanism that can alert a user as to changes in pivoting resistance that is made to the adjustable hinge 104.

As shown in FIG. 1B, the adjustment feature 105 of the adjustable hinge 104 can include a resistance control feature 115 that is configured to secure a pivoting resistance level of the adjustable hinge 104, such as a resistance level set by the user via the adjustment feature 105. For example, the resistance control feature 115 can include a plurality of grooves 116 that engage with a rotatable knob 123 and secure a rotational position of the rotatable knob 123 (e.g., corresponding to a resistance level). In some embodiments, a user can rotate the rotatable knob 123 to cause a change in pivoting resistance of the adjustable hinge 104. For example, the adjustment feature 105 can include a compressible spring 118 that assists with applying the compressive forces within the adjustable hinge 104 for increasing pivoting resistance between the first and second extensions 106a and 106b, respectively. For example, rotation of the rotatable knob 123 can result in an increase in compression of the spring 118 and one or more of the friction features 112, the first hinge-coupling end 114a, and the second hinge-coupling end 114b, thereby increasing compression within the adjustable hinge 104 and increase pivoting resistance between the first and second extensions 106a and 106b, respectively. The resistance control feature 115 can ensure a rotational position associated with a desired pivoting resistance level is maintained, including during use of the involuntary movement dampening device 100.

As shown in FIG. 1B, in some embodiments the first extension 106a and/or the second extension 106b can include one or more parts, such as one or more plate or retention fixtures 127 that can couple to at least one friction feature 112 for assisting with increasing and decreasing compressive forces within the adjustable hinge 104. One or both of the first extension 106a and the second extension 106b can include one or more parts without departing from the scope of this disclosure.

As shown in FIG. 1D, the second coupling feature 108b can include at least one nerve pressure feature 120 positioned along an inner wall 121 of the second coupling feature 108b. For example, the second coupling feature 108b can be configured to couple to an upper arm of a user and align the nerve pressure features 120 with the arm in order to apply a force against the ulnar nerve of the user. Such application of pressure against the ulnar nerve can assist with at least reducing pain and/or tremors experienced along the associated arm. As shown in FIG. 1D, the nerve pressure feature 120 can include an extruded material (e.g., rubber material) having one or more of a variety of shapes, such as a spherical shape. Other shapes of the nerve pressure feature 120 are within the scope of this disclosure.

Additionally, one or more nerve pressure features 120 can be positioned along the inner wall 121 of the first coupling feature 108a and/or second coupling feature 108b, such as two nerve pressure features 120 along the inner wall 121 of the second coupling feature 108b, as shown in FIG. 1D. The positioning and surface area of the one or more nerve pressure features 120 can be configured to avoid overstimulation of adjacent nerves while providing sufficient pressure to dampen nerve impulses of the ulnar nerve and/or radial nerve. The one or more nerve pressure features 120 can dampen nerve impulses to inhibit excessive movements in the condition being treated (e.g., tremors) and/or reduce pain associated with the associated extremity.

As shown in FIG. 1C, during use, the involuntary movement dampening device 100 can be aligned so the adjustable hinge 104 is positioned adjacent to a joint (e.g., elbow) of the user. For example, the axis of rotation of the adjustable hinge 104 can be either approximately parallel to or collinear with an axis of rotation of the adjacent joint. Additionally, the first coupling feature 108a can secure to a first part of an extremity associated with the joint (e.g., lower arm) and the second coupling feature can secure to a second part of the extremity (e.g., upper arm), or vice versa. Once both of the coupling features 108a and 108b are secured, the first extension 106a can extend along the first part of the extremity and act as a lever arm between the first coupling feature 108a and the adjustable hinge 104, and the second extension 106b can extend along the second part of the extremity and act as a lever arm between the second coupling feature 108b and the adjustable hinge 104. As such, increased pivoting resistance of the adjustable hinge 104 causes dampened involuntary movements of the associated extremity, such as between a lower arm and upper arm or lower leg and upper leg of the user.

As discussed above, the adjustable hinge 104 can provide various levels of pivoting resistance between the first and second extensions 106a and 106b, respectively, thereby at least dampening involuntary movements between the first and second parts of the extremity (e.g., dampen tremors and involuntary movements). Furthermore, one or more nerve pressure features 120 can extend from an inner wall 121 of the second coupling feature 108b and apply pressure along a back side of the upper arm in order to apply pressure against the associated ulnar nerve when the second coupling feature 108b is coupled to the upper arm. The involuntary movement dampening device 100 can be secured to a user in a variety of ways using one or more of a variety of coupling features, such as Velcro, straps, releasable locking mechanisms, etc., all of which are within the scope of this disclosure.

As discussed above, the involuntary movement dampening device 100 can be configured for use with a leg and/or arm, and can be adjustable for use with a variety of shaped and sized users (e.g., adults, children). As such, although FIG. 1C is shown as being configured for use with an arm of a user, any embodiment shown or described herein can be configured for use with a leg and/or arm of a user without departing from the scope of the present disclosure.

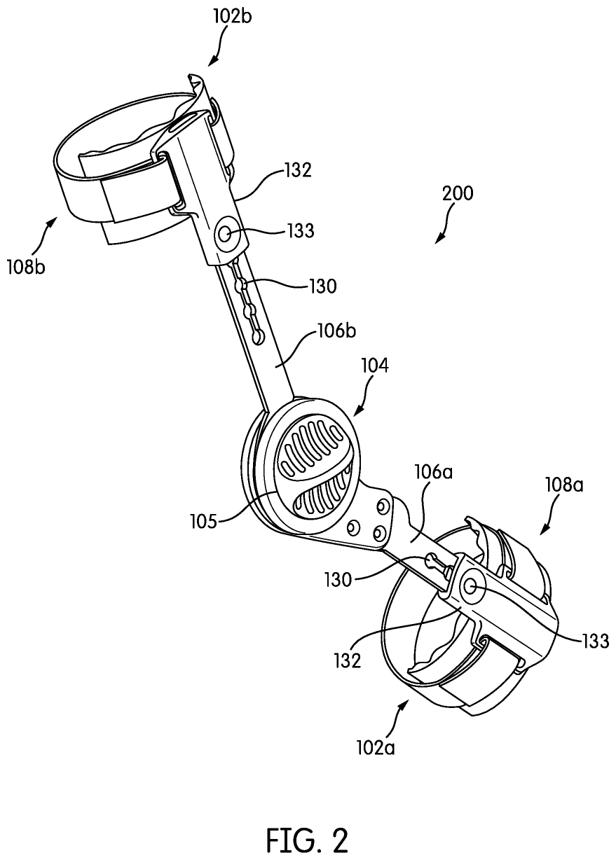

FIG. 2 illustrates another embodiment of the involuntary movement dampening device 200 including any one or more of the features described above with respect to the involuntary movement dampening device 100 of FIGS. 1A-1D. Additionally, as shown in FIG. 2, either the first coupling extension 102a or the second coupling extension 102b can include a length adjusting feature 130 that allows adjustments in length of a respective extension (e.g., first extension 106a or second extension 106b) such that the first coupling feature 108a and/or the second coupling feature 108b can be positioned closer and/or further away from the adjustable hinge 104. As such, the first coupling feature 108a and/or the second coupling feature 108b can be positioned at more than one distance from the adjustable hinge 104. This can allow the involuntary movement dampening device 200 to be configured to comfortably and effectively fit a variety of different sized users (e.g., children, adults). Furthermore, any number of a variety of length adjusting features 130 can be used to adjust the distance between each coupling feature 108a, 108b and the adjustable hinge 104.

For example, the length adjusting feature 130 can include a plurality of notched cutouts extending along the first extension 106a and/or the second extension 106b. For example, the length adjusting feature 130 along the first extension 106a can allow an engagement feature 133 (e.g., threaded securing element) to releasably secure a securing arm 132 of the first coupling feature 108a in a position relative to the adjustment hinge 104. For example, the securing arm 132 can be slidably engaged with the associated extension, such as the first extension 106a, such that sliding of the securing arm 132 along the associated extension moves the associated coupling feature, such as the first coupling feature 108a, towards or away from the adjustable hinge 104. For example, securing an engagement feature 133 through the securing arm 132 and length adjusting feature 130 can secure a distance between the coupling feature, such as the first coupling feature 108a, and the adjustment hinge 104. Other length adjusting features 130 are within the scope of this disclosure for adjusting the distance between each coupling feature 108a, 108b and the adjustable hinge 104.

FIGS. 3A-3B illustrate yet another embodiment of the involuntary movement dampening device 300 including an embodiment of the adjustable hinge 104 that includes a sensory feedback feature. For example, the sensory feedback feature can be felt by a user when the user adjusts the adjustable hinge 104, such as rotates an embodiment of the adjustment feature 105 shown in FIG. 3A to thereby change the pivoting resistance setting of the adjustable hinge 104. As shown in FIG. 3B, the adjustable hinge 104 can include a sensory feedback mechanism 140 that creates a perceptible feedback, such as a tactile feedback (e.g., change in turning resistance) and/or audible feedback (e.g., a “clicking” noise). Such perceptible feedback from the sensory feedback mechanism 140 can alert the user that a change in pivoting resistance has been made. This can allow a user to more efficiently and effectively change and set pivoting resistance settings, including without having to view the adjustable hinge 104. For example, a user can rotate the adjustment feature 105 (e.g., a first degree of rotation) thereby causing the sensory feedback mechanism 140 to rotate. As shown in FIG. 3B, the sensory feedback mechanism 140 can include at least one spring 142 coupled to or in contact with a spherical feature 143, which can be positioned between a base 144 and cap 145. For example, the sensory feedback mechanism 140 can be positioned within a housing 155 of the adjustment feature 105 and engage one or more contact features 156 (e.g., extrusions, indentations, etc.) of the housing 155 such that rotation of the housing 155 causes the sensory feedback mechanism 140 to actively engage with the one or more contact features 156, thereby causing sensory feedback to the user (e.g., sensed change in resistance of rotation of the housing 155, audible clicking noise, etc.). For example, the sensory feedback mechanism 140 can include at least one spherical feature that contacts and slides and/or rolls along a part of the housing 155 or other part of the adjustment feature 105. The spring 142 adjacent the spherical feature 143 can allow the spherical feature to move inward and outward, such as a result of interacting with features of the adjustment feature 105 (e.g., contact features 156 of the housing 155) during rotation of the adjustment feature, which can result in sensory feedback to the user (e.g., tactile and/or auditory feedback). Other sensory feedback mechanisms are within the scope of this disclosure for providing sensory feedback to a user when adjusting the adjustable hinge 104, such as to increase or decrease pivoting resistance.

As shown in FIG. 3A, one or both coupling extensions 102a, 102b can include one or more coupling features 108a, 108b, such as two coupling features associated with each coupling extension. In some embodiments, the first coupling extension 102a can include two first coupling features 108a and the second coupling extension 102b can include two second coupling extensions 108b, as shown in FIG. 3A. For example, the two first coupling features 108a can be at a fixed distance relative to each other along the associated first extension 106a. Additionally, an embodiment of the engagement feature 133 (e.g., depressible button, actuation feature, etc.) can be positioned along one or both coupling feature, such as to move at least one coupling feature along the associated extension. For example, one of the first coupling features 108a of the first coupling extension 102a can include one engagement feature 133 that, when disengaged with the first extension 106a, can allow the two first coupling features 108a to move simultaneously together for positioning along the first extension 106a relative to the adjustable hinge 104. In some embodiments, the two second coupling features 108b can include two coupling features 133, such as one engagement feature 133 for each of the two second coupling features 108b. As such, each engagement feature 133 can be independently engaged and disengaged, such as with a length adjusting feature 130, such as to allow positioning and movement of one of the second coupling features 108b along the second extension 106b relative to the adjustable hinge 104 and/or relative to the other second coupling feature 108b. For example, this can allow one of the second coupling features 108b including an embodiment of the nerve pressure feature 120 to be independently positioned along the second extension 106b, such as in order to properly position the nerve pressure feature 120 along the arm of a user for dampening nerve impulses of the ulnar nerve. Each engagement feature 133 can engage and disengage with a length adjusting feature 130 along the associated extension, such as the first extension 106a or the second extension 106b. Any number of a variety of engagement features 133 and length adjusting features 130 can be included in the first coupling extension 102a and/or second coupling extension 102b for allowing a plurality of configurations of one or more first coupling features 108a and one or more second coupling features 108b relative to each other and the adjustable hinge 104.

In some embodiments, and as shown in FIG. 3A, the housing 155 of the adjustment feature 105 can be configured to house and protect the adjustable hinge 104. As such, the housing 155 can be rotatable to adjust the pivoting resistance, as well as contain the resistance features (e.g., friction features 112), which can include one or more compressible or non-compressible materials positioned adjacent the first hinge-coupling end 114a and/or the second hinge-coupling end 114b for increasing friction therebetween to thereby dampen and control movement between the extensions 106a, 106b. The friction features 112 described herein can include one or more of a variety of material that can increase friction and dampen movement between two rotating flat members (e.g., first hinge-coupling end 114a and the second hinge-coupling end 114b) without departing from the scope of this disclosure.

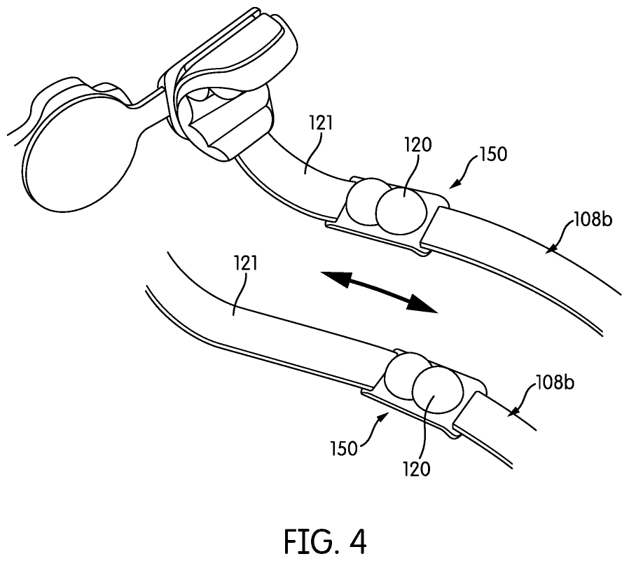

FIG. 4 illustrates an embodiment of the second coupling feature 108b including nerve pressure features 120 that are movable along the second coupling feature 108b. For example, nerve pressure features 120 can be coupled to a moveable element 150 that is slidably coupled to a part of the second coupling feature 108b such that the nerve pressure features 120 can be moved along the inner wall 121 of the second coupling feature 108b. This can allow a user to adjust placement of the nerve pressure features 120 along an inner wall 121 of the second coupling feature 108b for ensuring proper placement of the nerve pressure features 120 in order to provide pressure against the ulnar nerve and thereby assist with reducing pain and involuntary movement. In some embodiments, the nerve pressure features 120 can include one or more extruded features, such as two spherical shapes positioned in adjacent and/or in contact with each other. Various shaped and sized nerve pressure features 120 and movable elements 150 are within the scope of this disclosure.

An example method of use of an embodiment of the involuntary movement dampening device, such as any of the involuntary movement dampening device embodiments 100, 200, and 300 described herein, can include securing an embodiment of the involuntary movement dampening device 100 to an extremity of a user (e.g., arm of the user), such as using the first and second coupling features 108a, 108b. The method can further include adjusting a pivoting resistance of the adjustable hinge 104 of the involuntary movement dampening device 100 to achieve a second level of pivoting resistance, such that the second level of pivoting resistance is greater than the first level of pivoting resistance. The dampening of the involuntary movement can be greater as a result the adjustment hinge 104 having the second level of pivoting resistance compared to the adjustment hinge 104 having the first level of pivoting resistance. The adjustment hinge 104 can increase a compression force and friction force to at least one of a first hinge-coupling end 114a of the first extension 106a and/or a second hinge-coupling end 114b of the second extension 106b to thereby increase a pivoting resistance between the first extension 106a and the second extension 106b. The pivoting resistance level of the adjustment hinge 104 can be increased and decreased, as desired by the user, and a sensory feedback mechanism 140 of the adjustment hinge 104 can provide either auditory or tactile feedback to indicate changes in pivoting resistance levels (e.g., strength of pivoting resistance).

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. An involuntary movement dampening device, comprising:
a first coupling extension including a first extension and a first coupling feature positioned along the first extension, the first coupling feature configured to secure to a first part of an extremity of a user;
a second coupling extension including a second extension and a second coupling feature positioned along the second extension, the second coupling feature configured to secure to a second part of the extremity of the user;
an adjustable hinge that provides a pivotable coupling between the first extension and the second extension, the adjustable hinge providing more than one dampening settings, each dampening setting of the more than one dampening settings providing a different level of pivoting resistance between the first extension and the second extension to thereby at least dampen involuntary pivoting between the first part and the second part of the extremity.

2. The involuntary movement dampening device of claim 1, further comprising a nerve pressure feature positioned along the first coupling feature and/or the second coupling feature for applying a pressure against the ulnar nerve when the associated first coupling feature and/or the second coupling feature is coupled to the extremity.

3. The involuntary movement dampening device of claim 2, wherein the involuntary pivoting movement is caused by tremors or other involuntary movements experienced along the extremity of the user.

4. The involuntary movement dampening device of claim 2, wherein the nerve pressure feature includes an extruded material extending from an inner wall of the second coupling feature.

5. The involuntary movement dampening device of claim 4, wherein the extruded material includes a spherical shape.

6. The involuntary movement dampening device of claim 2, wherein the nerve pressure feature is moveable along an inner wall of the second coupling feature.

7. The involuntary movement dampening device of claim 1, further comprising at least one length adjusting feature for moving the first coupling feature or the second coupling feature closer to or further away from the adjustable hinge.

8. The involuntary movement dampening device of claim 1, wherein the adjustable hinge includes a friction feature that increases pivoting resistance between the first extension and the second extension as compression of the friction feature is increased within the adjustable hinge.

9. The involuntary movement dampening device of claim 8, wherein the friction feature decreases resistance in movement of the first and second extensions as compression of the friction feature decreases within the adjustable hinge.

10. The involuntary movement dampening device of claim 1, wherein the adjustable hinge is configured to provide at least three different levels of pivoting resistance between the first extension and the second extension.

11. The involuntary movement dampening device of claim 10, wherein the adjustable hinge includes an adjustment feature that can be rotated by a user to at least three different positions, each position configuring the adjustable hinge to provide one of the at least three different levels of pivoting resistance.

12. The involuntary movement dampening device of claim 11, wherein the adjustable hinge includes a sensory feedback mechanism that provides at least one of a tactile feedback and an audible feedback to a user as a result of the adjustable hinge changing to a different level of pivoting resistance.

13. The involuntary movement dampening device of claim 1, wherein the first coupling feature and the second coupling feature each include an adjustable strap and/or an inflatable strap.

14. The involuntary movement dampening device of claim 1, wherein the second coupling extension includes more than one second coupling feature that are moveable along the second extension relative to each other and relative to the adjustable hinge.

15. A method of an involuntary movement dampening device, comprising:
adjusting a pivoting resistance of an adjustable hinge of the involuntary movement dampening device to achieve a first level of pivoting resistance, the involuntary movement dampening device comprising:
a first coupling extension including a first extension and a first coupling feature positioned along the first extension, the first coupling feature configured to secure to a first part of an extremity of a user; and
a second coupling extension including a second extension and a second coupling feature positioned along the second extension, the second coupling feature configured to secure to a second part of the extremity of the user,
wherein the adjustable hinge provides a pivotable coupling between the first extension and the second extension, the adjustable hinge providing more than one dampening settings, each dampening setting of the more than one dampening settings providing a different level of pivoting resistance between the first extension and the second extension to thereby at least dampen involuntary pivoting between the first part and the second part of the extremity; and
dampening an involuntary movement of the extremity of the user, wherein the involuntary movement dampening device is coupled to the extremity.

16. The method of claim 15, wherein the involuntary movement dampening device includes a nerve pressure feature positioned along the first coupling feature and/or the second coupling feature for applying a pressure against the ulnar nerve when the associated first coupling feature and/or the second coupling feature is coupled to the extremity.

17. The method of claim 16, wherein the involuntary pivoting movement is caused by tremors or other involuntary movements experienced along the extremity of the user.

18. The method of claim 16, wherein the nerve pressure feature includes an extruded material extending from an inner wall of the second coupling feature.

19. The method of claim 18, wherein the extruded material includes a spherical shape.

20. The method of claim 16, wherein the nerve pressure feature is moveable along an inner wall of the second coupling feature.

21. The method of claim 15, wherein the involuntary movement dampening device includes at least one length adjusting feature for moving the first coupling feature or the second coupling feature closer to or further away from the adjustable hinge.

22. The method of claim 15, wherein the adjustable hinge includes a friction feature that increases pivoting resistance between the first extension and the second extension as compression of the friction feature is increased within the adjustable hinge.

23. The method of claim 22, wherein the friction feature decreases resistance in movement of the first and second extensions as compression of the friction feature decreases within the adjustable hinge.

24. The method of claim 15, wherein the adjustable hinge is configured to provide at least three different levels of pivoting resistance between the first extension and the second extension.

25. The method of claim 24, wherein the adjustable hinge includes an adjustment feature that can be rotated by a user to at least three different positions, each position configuring the adjustable hinge to provide one of the at least three different levels of pivoting resistance.

26. The method of claim 25, wherein the adjustable hinge includes a sensory feedback mechanism that provides at least one of a tactile feedback and an audible feedback to a user as a result of the adjustable hinge changing to a different level of pivoting resistance.

27.-31. (canceled)
